(19) United States
(12) Patent Application Publication
(10) Pub. No.: US 2007/0243192 A1
(43) Pub. Date: Oct. 18, 2007

(54) Growth hormone receptor antagonist cancer treatment

(75) Inventors: Max S. Wicha, Ann Arbor, MI (US); Gabriela Dontu, Ann Arbor, MI (US); Suling Liu, Ann Arbor, MI (US)

Correspondence Address: MEDLEN & CARROLL, LLP, 101 HOWARD STREET, SUITE 350, SAN FRANCISCO, CA, 94105, US

(73) Assignee: Regents of the University of Michigan, Ann Arbor, MI (US)

(21) Appl. No.: 11/708,895

(22) Filed: Feb. 21, 2007

Related U.S. Application Data
(60) Provisional application No. 60/775,129, filed on Feb. 21, 2006.

(51) Int. Cl. A61K 39/395; A61K 31/00; A61P 35/00; C12Q 1/02; G01N 33/574; C12N 1/00; A61K 38/00

(52) U.S. Cl. 424/138.1; 435/243; 435/7.23; 514/1; 514/12

(57) ABSTRACT

The present invention provides methods and compositions for treating tumorigenic cells (e.g., mammary progenitor cancer cells), with growth hormone receptor antagonists (e.g., Pegvisomant), as well as methods and compositions for screening growth hormone receptor antagonists for their ability serve as anti-neoplastic agents capable of killing tumorigenic cells. The present invention provides methods for identifying tumorigenic cells, methods of obtaining enriched populations of tumorigenic cells, and methods of causing mammary progenitor cells to proliferate and/or differentiate.

The present application claims priority to U.S. Provisional Application Ser. No. 60/775,129, filed Feb. 21, 2006, which is herein incorporated by reference.

The present invention was made with government support under grant number R01CA101860-02 from the National Institutes of Health. The government has certain rights in the invention.

### FIELD OF THE INVENTION

The present invention relates to methods and compositions for treating tumorigenic cells (e.g., mammary progenitor cancer cells), with growth hormone receptor antagonists (e.g., Pegvisomant), as well as methods and compositions for screening growth hormone receptor antagonists for their ability serve as anti-neoplastic agents capable of killing tumorigenic cells. The present invention also relates to methods for identifying tumorigenic cells, methods of obtaining enriched populations of tumorigenic cells, and methods of causing mammary progenitor cells to proliferate and/or differentiate.

### BACKGROUND

Cancer is one of the leading causes of death and metastatic cancer is often incurable. Although current therapies can produce tumor regression, they rarely cure common tumors such as metastatic breast cancer (Lippman, M. E., N Engl J Med 342, 1119-20 (2000), herein incorporated by reference). Solid tumors consist of heterogeneous populations of cancer cells. Like acute myeloid leukemia (AML) (Lapidot, T. et al., Nature 17, 645-648 (1994), herein incorporated by reference), it has been demonstrated recently that in most malignant human breast tumors, a small, distinct population of cancer cells are enriched for the ability to form tumors in immunodeficient mice (Al-Hajj et al., Proc Natl Acad Sci USA 100, 3983-8 (2003), herein incorporated by reference). Previously it was shown that in 8 of the 9 tumors studied, the CD44⁺CD24^{-/low}Lineage⁻ population had the ability to form tumors when injected into immunodeficient mice. As few as 200 of these cells, termed “tumorigenic” cells, consistently formed tumors in mice. In contrast, the majority of the cancer cells in a tumor consisted of “non-tumorigenic” cells with alternative phenotypes. These cells failed to form tumor in NOD/SCID mice even when as many as 10⁴ cells were injected (Al-Hajj et al, 2003). In some tumors further enrichment of the tumorigenic cells was possible by isolating the ESA⁺CD44⁺CD24^{-/low}Lineage⁻ population of cancer cells. What is needed therefore, are compositions and methods for treating tumorigenic cells (e.g. tumorigenic breast cancer cells), as well as methods for screening to identify such therapeutic compositions.

### SUMMARY OF THE INVENTION

The present invention provides methods and compositions for treating tumorigenic cells (e.g., mammary progenitor cancer cells), with growth hormone receptor antagonists (e.g., Pegvisomant), as well as methods and compositions for screening growth hormone receptor antagonists for their ability serve as anti-neoplastic agents capable of killing tumorigenic cells. The present invention provides methods for identifying tumorigenic cells, methods of obtaining enriched populations of tumorigenic cells, and methods of causing mammary progenitor cells to proliferate and/or differentiate.

In some embodiments, the present invention provides methods of reducing or eliminating tumorigenic cells in a subject (e.g. a subject that does not have acromegaly), comprising: administering a composition comprising Pegvisomant (e.g. SOMAVERT, Pfizer Inc.) to the subject. In other embodiments, the present invention provides methods for reducing or eliminating tumorigenic cells in a subject (e.g. a subject that does not have acromegaly), comprising: administering a growth hormone receptor antagonist to the subject (e.g., under conditions such that at least a portion of said tumorigenic cells are killed, inhibited from proliferating, and/or from causing metastasis). In certain embodiments, the present invention provides methods of treating a subject (e.g. a subject that does not have acromegaly) having a tumorigenic mammary cell, comprising administering a growth hormone receptor antagonist to the subject (e.g., under conditions such that at least a portion of said tumorigenic cells are killed, inhibited from proliferating, or from causing metastasis). In particular embodiments, the administering is under conditions such that the tumorigenic mammary cell is killed. In further embodiments, the present invention provides methods of preventing or reducing metastasis, comprising: administering a growth hormone receptor antagonist to a subject suspected of having metastasis.

In particular embodiments, the administering is conducted under conditions such that said tumorigenic cells are killed or inhibited from proliferating or causing metastasis. In certain embodiments, the tumorigenic cells are mammary progenitor cells characterized by an increased level of expression of growth hormone receptor compared to non-tumorigenic mammary cells from the subject (e.g. from the same tumor biopsy sample). In other embodiments, the tumorigenic cells are mammary progenitor cells. In further embodiments, the growth hormone receptor antagonist comprises an antibody or antibody fragment (e.g. specific for the growth hormone receptor produced in the subject or for the growth hormone produced in vivo by the subject). In some embodiments, the growth hormone receptor antagonist comprises a modified form of the human growth hormone protein (e.g. Pegvisomant and compounds described in U.S. Pat. No. 5,849,535, herein incorporated by reference).

In particular embodiments, the tumorigenic cells are mammary cells (or other types of tumorigenic cells) characterized by an increased level of expression (e.g. up-regulated) of growth hormone receptor (e.g., as compared to non-tumorigenic mammary cells from the subject). In some embodiments, the methods further comprise determining that the tumorigenic cells have an increased level of growth hormone receptor expression (e.g., as compared to non-tumorigenic cells from the subject). In certain embodiments, the tumorigenic or non-tumorigenic cells are mammary cells, cells of epithial origin, neoronal cells, pancreatic cells, colon cells, etc.).

In certain embodiments, the methods further comprise surgically removing a tumor from the subject prior to the administering step. In other embodiments, the administering further comprises providing a second agent to the subject, where the second agent is anti-neoplastic. In some embodiments, the administering is intravenous and is performed at a distance of no more than 10 inches from the tumorigneic breast cells (e.g. no more than 9, 8, 7, 6, 5, 4, 3, 2 or 1 inches from the targeted tumorigenic breast cells).

In further embodiments, the present invention provides methods for identifying the presence of a mammary progenitor cell in a sample, comprising: detecting increased expression of growth hormone receptor (GHR) in a cell in the sample, and identifying the cell as a mammary progenitor cell. In other embodiments, the present invention provides methods for identifying the presence of a tumorigenic cell in a tumor sample, comprising: detecting increased expression of growth hormone receptor (GHR) in a cell in the tumor sample, and identifying the cell as a tumorigenic cell.

In certain embodiments, the tumor sample comprises a breast cancer tumor sample. In other embodiments, the methods further comprise the step of selecting a treatment course of action for a subject based on the presence or absence of the tumorigenic cell in the tumor sample. In further embodiments, the treatment course of action comprises administration of a growth hormone receptor antagonist to the subject. Tumorigenic cells may be detected by any method. For example, detection of markers associated with tumorigenic cancer stem cells, as described, for example, in WO05005601 or co-pending U.S. application Ser. No. 10/864,207, both of which are herein incorporated by reference.

In particular embodiments, the present invention provides methods for screening a compound, comprising: a) exposing a sample comprising a tumorigenic cell (e.g. mammary cell) to a candidate anti-neoplastic compound, wherein the candidate anti-neoplastic compound comprises a growth hormone receptor antagonist; and b) detecting a change in the cell in response to the compound. In some embodiments, the sample comprises a non-adherent mammosphere. In certain embodiments, the growth hormone receptor antagonist comprises an antibody or antibody fragment. In further embodiments, the growth hormone receptor antagonist comprises a modified form of the human growth hormone protein. In particular embodiments, the sample comprises human breast tissue. In some embodiments, the detecting comprises detecting cell death of the tumorigenic breast cell. In further embodiments, the methods further comprise identifying the candidate anti-neoplastic agent as capable of killing tumorigenic cells.

In some embodiments, the present invention provides methods of obtaining an enriched population of progenitor cells, comprising a) providing an initial sample comprising progenitor and non-progenitor cells, and b) sorting the initial sample based on the growth hormone receptor (GHR) expression level in the cells such that an enriched population is generated, wherein the enriched population contains a higher percentage of progenitor cells than present in the initial sample. In certain embodiments, the sorting comprises the use of flow cytometry. In further embodiments, the sorting comprises the use of immuno-magnetic sorting. In other embodiments, the progenitor cells comprise tumorigenic cells and the non-progenitor cells comprise non-tumorigenic cells. In additional embodiments, the progenitor and non-progenitor cells comprise mammary cells.

In other embodiments, the present invention provides methods for expanding a mammary progenitor cell sample, comprising; a) providing a sample (e.g. isolated from an animal) comprising mammary progenitor cells, and b) treating the sample in vitro with growth hormone or a variant thereof under conditions such that the mammary progenitor cells proliferate, differentiate, or proliferate and differentiate. In particular embodiments, the sample comprises a non-adherent mammosphere.

In some embodiments, the present invention provides kits comprising; a) a composition comprising a growth hormone receptor antagonist; and b) an insert component comprising instructions for using the composition for treating breast cancer. In preferred embodiments, the growth hormone receptor antagonist comprises Pegvisomant (e.g. SOMAVERT, Pfizer inc.).

In certain embodiments, the present invention provides compositions comprising a growth hormone receptor antagonist and a second agent, wherein the second agent is known to reduce or eliminate breast cancer cells when administered to a subject.

### DESCRIPTION OF FIGURES

FIG. 1 shows results from Example 1, and specifically shows the effect of growth hormone (GH), growth hormone receptor antagonist (GHRA) and GH+GHRA treatments on mammosphere formation, with FIG. 1A showing representative microscopic fields of the mammosphere suspension cultures at low magnification, and FIG. 1B showing quantitative results of the experiment shown in 1A.

FIG. 2 shows the effect of growth hormone, estradiol and progesterone treatment on GH mRNA levels and Wnt mRNA levels as described in Example 1.

FIG. 3 shows the results from Example 2. FIG. 3A shows sections through mammospheres, immunostaining for ER(HRP-DAB enzymatic staining). FIG. 3B shows GH expression is up-regulated in mammospheres in response to estradiol and progesterone stimulations (mRNA levels compared by Q-RT-PCR). FIG. 3C shows conditioned medium from spheres treated with E2 and P increased increases sphere-formation in secondary passage

FIG. 4 shows the results from Example 3, and in particular shows the results of Pegvisomant treatment on tumor size in vivo over a number of days.

### DEFINITIONS

To facilitate an understanding of the present invention, a number of terms and phrases are defined below:

As used herein, the phrase “growth hormone receptor antagonist” includes any compound or agent that prevents growth hormone (GH) proteins from binding to or activating the growth hormone receptor (GHR) on cells. Examples of such compounds include anti-GH antibodies and anti-GHR antibodies, as well as modified forms of growth hormone such as Pegvisomant (e.g. SOMAVERT, Pfizer inc.). In certain preferred embodiments, modified forms of growth hormone are employed that can competitively bind to the growth hormone receptor (e.g., without triggering signal transduction via the growth hormone receptor).

As used herein, the terms “anticancer agent,” “conventional anticancer agent,” or “cancer therapeutic drug” refer to any therapeutic agents (e.g., chemotherapeutic compounds and/or molecular therapeutic compounds), radiation therapies, or surgical interventions, used in the treatment of cancer (e.g., in mammals).

As used herein, the terms “drug” and “chemotherapeutic agent” refer to pharmacologically active molecules that are used to diagnose, treat, or prevent diseases or pathological conditions in a physiological system (e.g., a subject, or in vivo, in vitro, or ex vivo cells, tissues, and organs). Drugs act by altering the physiology of a living organism, tissue, cell, or in vitro system to which the drug has been administered. It is intended that the terms “drug” and “chemotherapeutic agent” encompass anti-hyperproliferative and antineoplastic compounds as well as other biologically therapeutic compounds.

As used herein the term “prodrug” refers to a pharmacologically inactive derivative of a parent “drug” molecule that requires biotransformation (e.g., either spontaneous or enzymatic) within the target physiological system to release, or to convert (e.g., enzymatically, mechanically, electromagnetically, etc.) the “prodrug” into the active “drug.” “Prodrugs” are designed to overcome problems associated with stability, toxicity, lack of specificity, or limited bioavailability. Exemplary “prodrugs” comprise an active “drug” molecule itself and a chemical masking group (e.g., a group that reversibly suppresses the activity of the “drug”). Some preferred “prodrugs” are variations or derivatives of compounds that have groups cleavable under metabolic conditions. Exemplary “prodrugs” become pharmaceutically active in vivo or in vitro when they undergo solvolysis under physiological conditions or undergo enzymatic degradation or other biochemical transformation (e.g., phosphorylation, hydrogenation, dehydrogenation, glycosylation, etc.). Prodrugs often offer advantages of solubility, tissue compatibility, or delayed release in the mammalian organism. (See e.g., Bundgard, Design of Prodrugs, pp. 7-9, 21-24, Elsevier, Amsterdam (1985); and Silverman, The Organic Chemistry of Drug Design and Drug Action, pp. 352-401, Academic Press, San Diego, Calif. (1992)). Common “prodrugs” include acid derivatives such as esters prepared by reaction of parent acids with a suitable alcohol (e.g., a lower alkanol), amides prepared by reaction of the parent acid compound with an amine (e.g., as described above), or basic groups reacted to form an acylated base derivative (e.g., a lower alkylamide).

An “effective amount” is an amount sufficient to effect beneficial or desired results. An effective amount can be administered in one or more administrations.

As used herein, the term “administration” refers to the act of giving a drug, prodrug, antibody, or other agent, or therapeutic treatment to a physiological system (e.g., a subject or in vivo, in vitro, or ex vivo cells, tissues, and organs). Exemplary routes of administration to the human body can be through the eyes (opthalmic), mouth (oral), skin (transdermal), nose (nasal), lungs (inhalant), oral mucosa (buccal), ear, by injection (e.g., intravenously, subcutaneously, intratumorally, intraperitoneally, etc.) and the like.

“Coadministration” refers to administration of more than one chemical agent or therapeutic treatment (e.g., radiation therapy) to a physiological system (e.g., a subject or in vivo, in vitro, or ex vivo cells, tissues, and organs). “Coadministration” of the respective chemical agents (e.g. growth hormone receptor antagonist) and therapeutic treatments (e.g., radiation therapy) may be concurrent, or in any temporal order or physical combination.

As used herein, the term “bioavailability” refers to any measure of the ability of an agent to be absorbed into a biological target fluid (e.g., blood, cytoplasm, CNS fluid, and the like), tissue, organelle or intercellular space after administration to a physiological system (e.g., a subject or in vivo, in vitro, or ex vivo cells, tissues, and organs).

As used herein, the term “biodistribution” refers to the location of an agent in organelles, cells (e.g., in vivo or in vitro), tissues, organs, or organisms, after administration to a physiological system.

A “hyperproliferative disease,” as used herein refers to any condition in which a localized population of proliferating cells in an animal is not governed by the usual limitations of normal growth. Examples of hyperproliferative disorders include tumors, neoplasms, lymphomas and the like. A neoplasm is said to be benign if it does not undergo invasion or metastasis and malignant if it does either of these. A “metastatic” cell or tissue means that the cell can invade and destroy neighboring body structures. Hyperplasia is a form of cell proliferation involving an increase in cell number in a tissue or organ without significant alteration in structure or function. Metaplasia is a form of controlled cell growth in which one type of fully differentiated cell substitutes for another type of differentiated cell. Metaplasia can occur in epithelial or connective tissue cells. A typical metaplasia involves a somewhat disorderly metaplastic epithelium.

As used herein, the term “neoplastic disease” refers to any abnormal growth of cells or tissues being either benign (non-cancerous) or malignant (cancerous).

As used herein, the term “anti-neoplastic agent” refers to any compound that retards the proliferation, growth, or spread of a targeted (e.g., malignant) neoplasm.

As used herein, the term “regression” refers to the return of a diseased subject, cell, tissue, or organ to a non-pathological, or less pathological state as compared to basal nonpathogenic exemplary subject, cell, tissue, or organ. For example, regression of a tumor includes a reduction of tumor mass as well as complete disappearance of a tumor or tumors.

As used herein, the terms “prevent,” “preventing,” and “prevention,” in the context of regulation of hyper-proliferation, refer to a decrease in the occurrence of hyperproliferative or neoplastic cells in a subject. The prevention may be complete, e.g., the total absence of hyperproliferative or neoplastic cells in a subject. The prevention may also be partial, such that the occurrence of hyperproliferative or neoplastic cells in a subject is less than that which would have occurred without an intervention.

As used herein the term, “in vitro” refers to an artificial environment and to processes or reactions that occur within an artificial environment. In vitro environments can consist of, but are not limited to, test tubes and cell cultures. The term “in vivo” refers to the natural environment (e.g., an animal or a cell) and to processes or reactions that occur within a natural environment.

As used herein, the term “cell culture” refers to any in vitro culture of cells. Included within this term are continuous cell lines (e.g., with an immortal phenotype), primary cell cultures, finite cell lines (e.g., non-transformed cells), and any other cell population maintained in vitro, including oocytes and embryos.

As used herein, the term “subject” refers to organisms to be treated by the methods of the present invention. Such organisms include, but are not limited to, humans and veterinary animals (dogs, cats, horses, pigs, cattle, sheep, goats, and the like). In the context of the invention, the term “subject” generally refers to an individual who will receive or who has received treatment. In preferred embodiments, the subject does not have, or has not been diagnosed with, acromegaly.

The term “diagnosed,” as used herein, refers to the recognition of a disease by its signs and symptoms or genetic analysis, pathological analysis, histological analysis, and the like.

As used herein, the term “competes for binding” is used in reference to a first molecule with an activity that binds to the same target as does a second molecule. The efficiency (e.g., kinetics or thermodynamics) of binding by the first molecule may be the same as, or greater than, or less than, the efficiency of the target binding by the second molecule. For example, the equilibrium binding constant (Kd) for binding to the target may be different for the two molecules.

As used herein, the term “antisense” is used in reference to nucleic acid sequences (e.g., RNA, phosphorothioate DNA) that are complementary to a specific RNA sequence (e.g., mRNA). Included within this definition are natural or synthetic antisense RNA molecules, including molecules that regulate gene expression, such as small interfering RNAs or micro RNAs.

The term “test compound” or “candidate compound” refers to any chemical entity, pharmaceutical, drug, and the like, that can be used to treat or prevent a disease, illness, sickness, or disorder of bodily function, or otherwise alter the physiological or cellular status of a sample. Test compounds comprise both known and potential therapeutic compounds. A test compound can be determined to be therapeutic by using the screening methods of the present invention. A “known therapeutic compound” refers to a therapeutic compound that has been shown (e.g., through animal trials or prior experience with administration to humans) to be effective in such treatment or prevention. In preferred embodiments, “test compounds” are anticancer agents. In particularly preferred embodiments, “test compounds” are anticancer agents that induce apoptosis in cells.

As used herein, the term “antigen binding protein” refers to proteins which bind to a specific antigen. “Antigen binding proteins” include, but are not limited to, immunoglobulins, including polyclonal, monoclonal, chimeric, single chain, and humanized antibodies, Fab fragments, F(ab′)2 fragments, and Fab expression libraries. Preferably, the antigen binding proteins are specific for the human growth hormone receptor or human growth hormone. Various procedures known in the art are used for the production of polyclonal antibodies. For the production of antibodies, various host animals can be immunized by injection with the peptide corresponding to the desired epitope including, but not limited to, rabbits, mice, rats, sheep, goats, etc. In a preferred embodiment, the peptide is conjugated to an immunogenic carrier (e.g., diphtheria toxoid, bovine serum albumin (BSA), or keyhole limpet hemocyanin (KLH)). Various adjuvants are used to increase the immunological response, depending on the host species, including, but not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (Bacille Calmette-Guerin) and Corynebacterium parvum.

For preparation of monoclonal antibodies, any technique that provides for the production of antibody molecules by continuous cell lines in culture may be used (See e.g., Harlow and Lane, Antibodies: A Laboratory Manual, Cold Spring Harbor Laboratory Press, Cold Spring Harbor, N.Y.). These include, but are not limited to, the hybridoma technique originally developed by Köhler and Milstein (Köhler and Milstein, Nature, 256:495-497 (1975)), as well as the trioma technique, the human B-cell hybridoma technique (See e.g., Kozbor et al., Immunol. Today, 4:72 (1983)), and the EBV-hybridoma technique to produce human monoclonal antibodies (Cole et al., in Monoclonal Antibodies and Cancer Therapy, Alan R. Liss, Inc., pp. 77-96 (1985)).

According to the invention, techniques described for the production of single chain antibodies (U.S. Pat. No. 4,946,778; herein incorporated by reference) can be adapted to produce specific single chain antibodies as desired. An additional embodiment of the invention utilizes the techniques known in the art for the construction of Fab expression libraries (Huse et al., Science, 246:1275-1281 (1989)) to allow rapid and easy identification of monoclonal Fab fragments with the desired specificity.

Antibody fragments that contain the idiotype (antigen binding region) of the antibody molecule can be generated by known techniques. For example, such fragments include, but are not limited to: the F(ab′)2 fragment that can be produced by pepsin digestion of an antibody molecule; the Fab′ fragments that can be generated by reducing the disulfide bridges of an F(ab′)2 fragment, and the Fab fragments that can be generated by treating an antibody molecule with papain and a reducing agent.

Genes encoding antigen-binding proteins can be isolated by methods known in the art. In the production of antibodies, screening for the desired antibody can be accomplished by techniques known in the art (e.g., radioimmunoassay, ELISA (enzyme-linked immunosorbant assay), “sandwich” immunoassays, immunoradiometric assays, gel diffusion precipitin reactions, immunodiffusion assays, in situ immunoassays (using colloidal gold, enzyme or radioisotope labels, for example), Western Blots, precipitation reactions, agglutination assays (e.g., gel agglutination assays, hemagglutination assays, etc.), complement fixation assays, immunofluorescence assays, protein A assays, and immunoelectrophoresis assays, etc.) etc.

As used herein, the term “modulate” refers to the activity of a compound to affect (e.g., to promote or retard) an aspect of the cellular function including, but not limited to, cell growth, proliferation, invasion, angiogenesis, apoptosis, and the like.

### DETAILED DESCRIPTION OF THE INVENTION

The present invention provides methods and compositions for treating tumorigenic cells (e.g., mammary progenitor cancer cells), with growth hormone antagonists (e.g., Pegvisomant), as well as methods and compositions for screening growth hormone antagonists for their ability to serve as anti-neoplastic agents capable of killing tumorigenic cells. The present invention provides methods for identifying tumorigenic cells, methods of obtaining enriched populations of tumorigenic cells, and methods of causing mammary progenitor cells to proliferate and/or differentiate.

As described in Example 1 below, utilizing molecular profiling, it was determined that normal human mammary stem cells have increased expression of receptors for growth hormone compared to their differentiated counterparts. This was determined by culturing primitive human mammary cells as mammospheres. It was also determined that growth hormone is able to potentiate the self-renewal of these primitive mammary cells, and that this process is inhibited by the growth hormone receptor antagonists such as Pegvisomant (e.g., SOMAVERT, Pfizer inc.). Pegvisomant specifically competes with growth hormone for binding to the growth hormone receptor. It was further determined, by real time PCR, that growth hormone is produced by both normal and tumorigenic breast stem cells. Further results demonstrated, by real time PCR, that the production of growth hormone by these cells is regulated by the hormones estrogen and progesterone. It was also found that tumor stem cells identified as having the phenotype CD44+ CD24 Low Lin− have increased expression of growth hormone receptors compared to non-tumorigenic cells derived from the same tumor. While not limited to any mechanism, and not necessary to practice the present invention, it is contemplated that in breast carcinogenesis, tumor stem cells are driven by paracrine production of growth hormone, with this hormone acting in an autocrine or paracrine manner on breast cancer stem cells regulating their self-renewal. As such, the present invention provides methods for administering growth hormone receptor antagonists, such as Pegvisomant, for inhibiting self-renewal of breast cancer stem cells.

I. Tumorigenic Cancer Cells

Solid tumors consist of heterogeneous populations of cancer cells that differ in their ability to form new tumors. Cancer cells that have the ability to form tumors (i.e., tumorigenic cancer cells) and cancer cells that lack this capacity (i.e., non-tumorigenic cancer cells) can be distinguished based on phenotype (Al-Hajj, et al., Proc Natl Acad Sci USA 100, 3983-8 (2003); Pat. Pub. 20020119565; Pat. Pub. 20040037815; Pat. Pub. 20050232927; WO05/005601; Pat. Pub. 20050089518; U.S. application Ser. No. 10/864,207; Al-Hajj et al., Oncogene, 2004, 23:7274; and Clarke et al., Ann Ny Acad. Sci., 1044:90, 2005, all of which are herein incorporated by reference in their entireties for all purposes).

The present invention relates to compositions and methods for characterizing, regulating, diagnosing, and treating cancer. For example, the present invention provides compositions and methods for inhibiting tumorigenesis of certain classes of cancer cells, including breast cancer cells and preventing metastasis (e.g., using growth hormone receptor antagonists). The present invention also provides systems and methods for identifying compounds that regulate tumorigenesis. For example, the present invention provides methods for identifying tumorigenic cells and diagnosing diseases (e.g., hyperproliferative diseases) or biological events (e.g., tumor metastasis) associated with the presence of tumorigenic cells. In particular, the present invention identifies classes of cells within cancers that are tumorigenic and provides detectable characteristics of such cells (e.g. up regulated expression of growth hormone receptor), such that their presence can be determined, for example, in choosing whether to submit a subject to a medical intervention, selecting an appropriate treatment course of action, monitoring the success or progress of a therapeutic course of action (e.g., in a drug trial or in selecting individualized, ongoing therapy), or screening for new therapeutic compounds or therapeutic targets.

In some embodiments, the expression of growth hormone receptor proteins and/or regulators of the growth hormone signaling pathways is used to identify tumorigenic cells. Regulators of growth hormone receptor proteins and growth hormone signaling pathways also find use in research, drug screening, and therapeutic methods. For example, growth hormone receptor antagonists and antagonists of growth hormone signaling pathways find use in preventing or reducing cell proliferation, hyperproliferative disease development or progression, and cancer metastasis. In some embodiments, antagonists are utilized following removal of a solid tumor mass to help reduce proliferation and metastasis of remaining hyperproliferative cells.

The present invention is not limited to any particular type of tumorigenic cell type, nor is the present invention limited by the nature of the compounds or factors used to regulate tumorigenesis. Thus, while the present invention is illustrated below using breast cancer cells, skilled artisans will appreciate that the present invention is not limited to these illustrative examples. For example, it is contemplated that are variety of neoplastic conditions benefit from the teachings of the present invention, including, but not limited to, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma.

The observation that tumors contain a small population of tumorigenic cells with a common cell surface phenotype (e.g. up-regulated expression of growth hormone receptor) has important implications for understanding solid tumor biology and also for the development of effective cancer therapies. The inability of current cancer treatments to cure metastatic disease may be due to ineffective killing of tumorigenic cells. If the tumorigenic cells are spared by an agent, then tumors may regress but the remaining tumorigenic cells will drive tumor recurrence. By focusing on the tumorigenic population, one can identify and affect critical proteins involved in essential biological functions in the tumorigenic population of cancer cells, such as self-renewal and survival.

II. Growth Hormone Variants and Growth Hormone Receptor Antagonists

The methods and compositions of the present invention contemplate the use of growth hormone (GH), as well as variants of growth hormone that have activity similar (or superior) to growth hormone. Preferably the GH is human GH, although the present invention is not limited to GH (or variant thereof) from any particular species. In certain embodiments, the GH or variant thereof is used to cause the proliferation, differentiation, or proliferation and differentiation of progenitor cells, such as mammary progenitor cells.

Human growth hormone (hGH) is a protein of 191 amino acids secreted by the pituitary. Human growth hormone (hGH) participates in much of the regulation of normal human growth and development. This 22,000-dalton pituitary hormone exhibits a multitude of biological effects, including linear growth (somatogenesis), lactation, activation of macrophages, and insulin-like and diabetogenic effects, among others (Chawla, Annu. Rev. Med., 34: 519 (1983); Edwards et al., Science, 239: 769 (1988); Isaksson et al., Annu. Rev. Physiol., 47: 483 (1985); Thomer and Vance, J. Clin. Invest., 82: 745 (1988); Hughes and Friesen, Annu. Rev. Physiol., 47: 469 (1985)). These biological effects derive from the interaction between hGH and specific cellular receptors. The primary target for growth hormone is the growth hormone receptor found on cells throughout the body. The receptors are activated by hormone-induced dimerization resulting in cell signaling primarily through the JAK2/STAT3 pathway. hGH causes stimulation of tissue growth (especially muscle, bone and cartilage), increased cell growth, metabolism and differentiation. Much of hGH activity is achieved indirectly by hGH stimulation of IGF-I secretion.

In certain embodiments, the methods and compositions of the present invention employed a variant of growth hormone. Examples of such variants include, but are not limited to, truncated versions of the full length growth hormone protein, and mutated versions with substitutions and/or deletions. Exemplary variants may be found in the following patents: U.S. Pat. Nos. 5,849,535; 5,854,026; 6,022,711; 5,834,598; 6,143,523; 5,534,617; 5,750,373; 4,670,393; 5,688,666; and 6,136,563, all of which are herein incorporated by reference in their entireties.

The methods and compositions of the present invention also contemplate the use of growth hormone receptor antagonists such as Pegvisomant, as well as growth hormone receptor antagonists with similar (or increased) anti-tumorigenic activity as Pegvisomant. Exemplary growth hormone receptor antagonists include, but are not limited to, octapeptide, somatostatin, analoguem, lanreotide, angiopeptin, dermopeptin, and octreotide. Additional guidance on designing and therapeutically using such compounds may be found in the following references: Fuh et al., Rational design of potent antagonists to the human growth hormone receptor, Science. 1992; 256:1677-1680; Herman-Bonert et al., Growth hormone receptor antagonist therapy in acromegalic patients resistant to somatostatin analogs, J Clin Endocrinol Metab. 2000; 85:2958-2961; and Trainer et al., Treatment of acromegaly with the growth hormone-receptor antagonist pegvisomant, N. Engl J. Med. 2000; 342:1171-1177; all of which are herein incorporated by reference in their enteritis.

Exemplary growth hormone receptor antagonists are provided in the following patents: U.S. Pat. Nos. 6,936,440; 6,583,115; and 5,849,535, all of which are herein incorporated by reference in their entireties. In some embodiments, the growth hormone receptor antagonist comprises Pegvisomant, and specifically SOMAVERT (called injectable Pegvisomant) from Pfizer.

III. Non-Adherent Mammospheres and Antagonist Screening

In certain embodiments, the present invention employs non-adherent mammospheres for various screening procedures, including; methods for screening growth hormone receptor antagonists (e.g. to determine if they have similar activity to Pegvisomant), and screening growth hormone variants do determine if they have similar activity to growth hormone (e.g. to determine if they are able to cause proliferation and/or differentiation of progenitor cells, such as mammary progenitor cells).

Non-adherent mammospheres are an in vitro culture system that allows for the propagation of primary human mammary epithelial stem and progenitor cells in an undifferentiated state, based on their ability to proliferate in suspension as spherical structures. Non-adherent mammospheres have previously been described in Dontu et al Genes Dev. 2003 May 15; 17(10):1253-70, and Dontu et al., Breast Cancer Res. 2004; 6(6):R605-15. These references are incorporated by reference in their entireties and specifically for teaching the construction and use of non-adherent mammospheres. As described in Dontu et al., mammospheres have been characterized as being composed of stem and progenitor cells capable of self-renewal and multi-lineage differentiation. Dontu et al. also describes that mammospheres contain cells capable of clonally generating complex functional ductal-alveolar structures in reconstituted 3-D culture systems in Matrigel.

IV. Therapeutic Compositions and Administration

A pharmaceutical composition containing a regulator of tumorigenesis according the present invention can be administered by any effective method. For example, a growth hormone receptor antagonist, or other therapeutic agent that acts as an agonist or antagonist of proteins in the growth hormone signal transduction/response pathway can be administered by any effective method. For example, a physiologically appropriate solution containing an effective concentration of a growth hormone receptor antagonist can be administered topically, intraocularly, parenterally, orally, intranasally, intravenously, intramuscularly, subcutaneously or by any other effective means. In particular, the growth hormone receptor antagonist agent may be directly injected into a target cancer or tumor tissue by a needle in amounts effective to treat the tumor cells of the target tissue. Alternatively, a cancer or tumor present in a body cavity such as in the eye, gastrointestinal tract, genitourinary tract (e.g., the urinary bladder), pulmonary and bronchial system and the like can receive a physiologically appropriate composition (e.g., a solution such as a saline or phosphate buffer, a suspension, or an emulsion, which is sterile) containing an effective concentration of a growth hormone receptor antagonist via direct injection with a needle or via a catheter or other delivery tube placed into the cancer or tumor afflicted hollow organ. Any effective imaging device such as X-ray, sonogram, or fiber-optic visualization system may be used to locate the target tissue and guide the needle or catheter tube. In another alternative, a physiologically appropriate solution containing an effective concentration of a growth hormone receptor antagonist can be administered systemically into the blood circulation to treat a cancer or tumor that cannot be directly reached or anatomically isolated.

Such manipulations have in common the goal of placing the growth hormone receptor antagonist in sufficient contact with the target tumor to permit the growth hormone receptor antagonist to contact, transduce or transfect the tumor cells (depending on the nature of the agent). In one embodiment, solid tumors present in the epithelial linings of hollow organs may be treated by infusing the suspension into a hollow fluid filled organ, or by spraying or misting into a hollow air filled organ. Thus, the tumor cells (such as a solid tumor stem cells) may be present in or among the epithelial tissue in the lining of pulmonary bronchial tree, the lining of the gastrointestinal tract, the lining of the female reproductive tract, genitourinary tract, bladder, the gall bladder and any other organ tissue accessible to contact with the growth hormone receptor antagonist. In another embodiment, the solid tumor may be located in or on the lining of the central nervous system, such as, for example, the spinal cord, spinal roots or brain, so that the growth hormone receptor antagonist infused in the cerebrospinal fluid contacts and transduces the cells of the solid tumor in that space. One skilled in the art of oncology can appreciate that the growth hormone receptor antagonist can be administered to the solid tumor by direct injection into the tumor so that the growth hormone receptor antagonist contacts and affects the tumor cells inside the tumor.

The tumorigenic cells identified by the present invention can also be used to raise anti-cancer cell antibodies. In one embodiment, the method involves obtaining an enriched population of tumorigenic cells or isolated tumorigenic cells; treating the population to prevent cell replication (for example, by irradiation); and administering the treated cell to a human or animal subject in an amount effective for inducing an immune response to solid tumor stem cells. For guidance as to an effective dose of cells to be injected or orally administered; see, U.S. Pat. Nos. 6,218,166, 6,207,147, and 6,156,305, incorporated herein by reference. In another embodiment, the method involves obtaining an enriched population of solid tumor stem cells or isolated solid tumor stem cells; mixing the tumor stem cells in an in vitro culture with immune effector cells (according to immunological methods known in the art) from a human subject or host animal in which the antibody is to be raised; removing the immune effector cells from the culture; and transplanting the immune effector cells into a host animal in a dose that is effective to stimulate an immune response in the animal.

In some embodiments, the therapeutic agent is an antibody (e.g., an anti-GHR antibody). Monoclonal antibodies to may be prepared using any technique which provides for the production of antibody molecules by continuous cell lines in culture. These include, but are not limited to, the hybridoma technique, the human B-cell hybridoma technique, and the EBV-hybridoma technique (see, e.g., Kozbor, D. et al., J. Immunol. Methods 81:31-42 (1985); Cote R J et al. Proc. Natl. Acad. Sci. 80:2026-2030 (1983); and Cole S P et al. Mol. Cell. Biol. 62:109-120 (1984)).

In addition, techniques developed for the production of “chimeric antibodies,” such as the splicing of mouse antibody genes to human antibody genes to obtain a molecule with appropriate antigen specificity and biological activity, can be used (see, e.g., Morrison S L et al. Proc. Natl. Acad. Sci. 81:6851-6855 (1984); Neuberger M S et al. Nature 312:604-608 (1984); and Takeda S et al. Nature 314:452-454 (1985), both of which are herein incorporated by reference).

Various immunoassays may be used for screening to identify antibodies having the desired specificity. Numerous protocols for competitive binding or immunoradiometric assays using either polyclonal or monoclonal antibodies with established specificities are well known in the art. The antibody can also be a humanized antibody. Antibodies are humanized so that they are less immunogenic and therefore persist longer when administered therapeutically to a patient.

Human antibodies can be generated using the XENOMOUSE technology from Abgenix (Fremont, Calif., USA), which enables the generation and selection of high affinity, fully human antibody product candidates to essentially any disease target appropriate for antibody therapy. See, U.S. Pat. Nos. 6,235,883; 6,207,418; 6,162,963; 6,150,584; 6,130,364; 6,114,598; 6,091,001; 6,075,181; 5,998,209; 5,985,615; 5,939,598; and 5,916,771, each incorporated by reference; Yang X et al., Crit Rev Oncol Hemato 38(1): 17-23 (2001); Chadd H E & Chamow S M. Curr Opin Biotechnol 12(2):188-94 (2001); Green L L, Journal of Immunological Methods 231 11-23 (1999); Yang X-D et al., Cancer Research 59(6): 1236-1243 (1999); and Jakobovits A, Advanced Drug Delivery Reviews 31: 33-42 (1998). Antibodies with fully human protein sequences are generated using genetically engineered strains of mice in which mouse antibody gene expression is suppressed and functionally replaced with human antibody gene expression, while leaving intact the rest of the mouse immune system.

In some embodiments of the present invention, the anti-tumorigenic therapeutic agents (e.g. growth hormone receptor antagonists) of the present invention are co-adminstered with other anti-neoplastic therapies. A wide range of therapeutic agents find use with the present invention. Any therapeutic agent that can be co-administered with the agents of the present invention, or associated with the agents of the present invention is suitable for use in the methods of the present invention.

Some embodiments of the present invention provide methods (therapeutic methods, research methods, drug screening methods) for administering a therapeutic compound of the present invention and at least one additional therapeutic agent (e.g., including, but not limited to, chemotherapeutic antineoplastics, antimicrobials, antivirals, antifungals, and anti-inflammatory agents) and/or therapeutic technique (e.g., surgical intervention, radiotherapies).

Various classes of antineoplastic (e.g., anticancer) agents are contemplated for use in certain embodiments of the present invention. Anticancer agents suitable for use with the present invention include, but are not limited to, agents that induce apoptosis, agents that inhibit adenosine deaminase function, inhibit pyrimidine biosynthesis, inhibit purine ring biosynthesis, inhibit nucleotide interconversions, inhibit ribonucleotide reductase, inhibit thymidine monophosphate (TMP) synthesis, inhibit dihydrofolate reduction, inhibit DNA synthesis, form adducts with DNA, damage DNA, inhibit DNA repair, intercalate with DNA, deaminate asparagines, inhibit RNA synthesis, inhibit protein synthesis or stability, inhibit microtubule synthesis or function, and the like.

In some embodiments, exemplary anticancer agents suitable for use in compositions and methods of the present invention include, but are not limited to: 1) alkaloids, including microtubule inhibitors (e.g., vincristine, vinblastine, and vindesine, etc.), microtubule stabilizers (e.g., paclitaxel (TAXOL), and docetaxel, etc.), and chromatin function inhibitors, including topoisomerase inhibitors, such as epipodophyllotoxins (e.g., etoposide (VP-16), and teniposide (VM-26), etc.), and agents that target topoisomerase I (e.g., camptothecin and isirinotecan (CPT-11), etc.); 2) covalent DNA-binding agents (alkylating agents), including nitrogen mustards (e.g., mechlorethamine, chlorambucil, cyclophosphamide, ifosphamide, and busulfan (MYLERAN), etc.), nitrosoureas (e.g., carmustine, lomustine, and semustine, etc.), and other alkylating agents (e.g., dacarbazine, hydroxymethylmelamine, thiotepa, and mitomycin, etc.); 3) noncovalent DNA-binding agents (antitumor antibiotics), including nucleic acid inhibitors (e.g., dactinomycin (actinomycin D), etc.), anthracyclines (e.g., daunorubicin (daunomycin, and cerubidine), doxorubicin (adriamycin), and idarubicin (idamycin), etc.), anthracenediones (e.g., anthracycline analogues, such as mitoxantrone, etc.), bleomycins (BLENOXANE), etc., and plicamycin (mithramycin), etc.; 4) antimetabolites, including antifolates (e.g., methotrexate, FOLEX, and MEXATE, etc.), purine antimetabolites (e.g., 6-mercaptopurine (6-MP, PURINETHOL), 6-thioguanine (6-TG), azathioprine, acyclovir, ganciclovir, chlorodeoxyadenosine, 2-chlorodeoxyadenosine (CdA), and 2′-deoxycoformycin (pentostatin), etc.), pyrimidine antagonists (e.g., fluoropyrimidines (e.g., 5-fluorouracil (ADRUCIL), 5-fluorodeoxyuridine (FdUrd) (floxuridine)) etc.), and cytosine arabinosides (e.g., CYTOSAR (ara-C) and fludarabine, etc.); 5) enzymes, including L-asparaginase, and hydroxyurea, etc.; 6) hormones, including glucocorticoids, antiestrogens (e.g., tamoxifen, etc.), nonsteroidal antiandrogens (e.g., flutamide, etc.), and aromatase inhibitors (e.g., anastrozole (ARIMIDEX), etc.); 7) platinum compounds (e.g., cisplatin and carboplatin, etc.); 8) monoclonal antibodies conjugated with anticancer drugs, toxin:, and/or radionuclides, etc.; 9) biological response modifiers (e.g., interferons (e.g., IFN-α, etc.) and interleukins (e.g., IL-2, etc.), etc.); 10) adoptive immunotherapy; 11) hematopoietic growth factors; 12) agents that induce tumor cell differentiation (e.g., all-trans-retinoic acid, etc.); 13) gene therapy techniques; 14) antisense therapy techniques; 15) tumor vaccines; 16) therapies directed against tumor metastases (e.g., batimastat, etc.); 17) angiogenesis inhibitors; 18) proteosome inhibitors (e.g., VELCADE); 19) inhibitors of acetylation and/or methylation (e.g., HDAC inhibitors); 20) modulators of NF kappa B; 21) inhibitors of cell cycle regulation (e.g., CDK inhibitors); 22) modulators of p53 protein function; and 23) radiation.

Any oncolytic agent that is routinely used in a cancer therapy context finds use in the compositions and methods of the present invention. For example, the U.S. Food and Drug Administration maintains a formulary of oncolytic agents approved for use in the United States. International counterpart agencies to the U.S.F.D.A. maintain similar formularies. Table 1 provides a list of exemplary antineoplastic agents approved for use in the U.S. Those skilled in the art will appreciate that the “product labels” required on all U.S. approved chemotherapeutics describe approved indications, dosing information, toxicity data, and the like, for the exemplary agents.

TABLE 1

| | | |
|---|---|---|
| Aldesleukin | Proleukin | Chiron Corp., |
| (des-alanyl-1, serine-125 human interleukin-2) | | Emeryville, CA |
| Alemtuzumab | Campath | Millennium and |
| (IgG1κ anti CD52 antibody) | | ILEX Partners, LP, |
| | | Cambridge, MA |
| Alitretinoin | Panretin | Ligand |
| (9-cis-retinoic acid) | | Pharmaceuticals, Inc., |
| | | San Diego CA |
| Allopurinol | Zyloprim | GlaxoSmithKline, |
| (1,5-dihydro-4H-pyrazolo[3,4-d]pyrimidin-4-one | | Research Triangle |
| monosodium salt) | | Park, NC |
| Altretamine | Hexalen | US Bioscience, West |
| (N,N,N′,N′,N″,N″,-hexamethyl-1,3,5-triazine-2,4, | | Conshohocken, PA |
| 6-triamine) | | |
| Amifostine | Ethyol | US Bioscience |
| (ethanethiol, 2-[(3-aminopropyl)amino]-, | | |
| dihydrogen phosphate (ester)) | | |
| Anastrozole | Arimidex | AstraZeneca |
| (1,3-Benzenediacetonitrile,a,a,a′,a′-tetramethyl- | | Pharmaceuticals, LP, |
| 5-(1H-1,2,4-triazol-1-ylmethyl)) | | Wilmington, DE |
| Arsenic trioxide | Trisenox | Cell Therapeutic, |
| | | Inc., Seattle, WA |
| Asparaginase | Elspar | Merck & Co., Inc., |
| (L-asparagine amidohydrolase, type EC-2) | | Whitehouse Station, |
| | | NJ |
| BCG Live | TICE BCG | Organon Teknika, |
| (lyophilized preparation of an attenuated strain of | | Corp., Durham, NC |
| Mycobacterium bovis (Bacillus Calmette-Gukin | | |
| [BCG], substrain Montreal) | | |
| bexarotene capsules | Targretin | Ligand |
| (4-[1-(5,6,7,8-tetrahydro-3,5,5,8,8-pentamethyl-2- | | Pharmaceuticals |
| napthalenyl) ethenyl] benzoic acid) | | |
| bexarotene gel | Targretin | Ligand |
| | | Pharmaceuticals |
| Bleomycin | Blenoxane | Bristol-Myers Squibb |
| (cytotoxic glycopeptide antibiotics produced by | | Co., NY, NY |
| Streptomyces verticillus; bleomycin A₂ and | | |
| bleomycin B₂) | | |
| Capecitabine | Xeloda | Roche |
| (5′-deoxy-5-fluoro-N-[(pentyloxy)carbonyl]- | | |
| cytidine) | | |
| Carboplatin | Paraplatin | Bristol-Myers Squibb |
| (platinum, diammine [1,1- | | |
| cyclobutanedicarboxylato(2-)-0,0′]-,(SP-4-2)) | | |
| Carmustine | BCNU, BiCNU | Bristol-Myers Squibb |
| (1,3-bis(2-chloroethyl)-1-nitrosourea) | | |
| Carmustine with Polifeprosan 20 Implant | Gliadel Wafer | Guilford |
| | | Pharmaceuticals, Inc., |
| | | Baltimore, MD |
| Celecoxib | Celebrex | Searle |
| (as 4-[5-(4-methylphenyl)-3-(trifluoromethyl)- | | Pharmaceuticals, |
| 1H-pyrazol-1-yl] | | England |
| benzenesulfonamide) | | |
| Chlorambucil | Leukeran | GlaxoSmithKline |
| (4-[bis(2chlorethyl)amino]benzenebutanoic acid) | | |
| Cisplatin | Platinol | Bristol-Myers Squibb |
| (PtCl₂H₆N₂) | | |
| Cladribine | Leustatin, 2-CdA | R.W. Johnson |
| (2-chloro-2′-deoxy-b-D-adenosine) | | Pharmaceutical |
| | | Research Institute, |
| | | Raritan, NJ |
| Cyclophosphamide | Cytoxan, Neosar | Bristol-Myers Squibb |
| (2-[bis(2-chloroethyl)amino] tetrahydro-2H-13,2- | | |
| oxazaphosphorine 2-oxide monohydrate) | | |
| Cytarabine | Cytosar-U | Pharmacia & Upjohn |
| (1-b-D-Arabinofuranosylcytosine, C₉H₁₃N₃O₅) | | Company |
| cytarabine liposomal | DepoCyt | Skye |
| | | Pharmaceuticals, Inc., |
| | | San Diego, CA |
| Dacarbazine | DTIC-Dome | Bayer AG, |
| (5-(3,3-dimethyl-1-triazeno)-imidazole-4- | | Leverkusen, |
| carboxamide (DTIC)) | | Germany |
| Dactinomycin, actinomycin D | Cosmegen | Merck |
| (actinomycin produced by Streptomyces parvullus, | | |
| C₆₂H₈₆N₁₂O₁₆) | | |
| Darbepoetin alfa | Aranesp | Amgen, Inc., |
| (recombinant peptide) | | Thousand Oaks, CA |
| daunorubicin liposomal | DanuoXome | Nexstar |
| ((8S-cis)-8-acetyl-10-[(3-amino-2,3,6-trideoxy-a- | | Pharmaceuticals, Inc., |
| L-lyxo-hexopyranosyl)oxy]-7,8,9,10-tetrahydro- | | Boulder, CO |
| 6,8,11-trihydroxy-1-methoxy-5,12- | | |
| naphthacenedione hydrochloride) | | |
| Daunorubicin HCl, daunomycin | Cerubidine | Wyeth Ayerst, |
| ((1S,3S)-3-Acetyl-1,2,3,4,6,11-hexahydro- | | Madison, NJ |
| 3,5,12-trihydroxy-10-methoxy-6,11-dioxo-1- | | |
| naphthacenyl 3-amino-2,3,6-trideoxy-(alpha)-L- | | |
| lyxo-hexopyranoside hydrochloride) | | |
| Denileukin diftitox | Ontak | Seragen, Inc., |
| (recombinant peptide) | | Hopkinton, MA |
| Dexrazoxane | Zinecard | Pharmacia & Upjohn |
| ((S)-4,4′-(1-methyl-1,2-ethanediyl)bis-2,6- | | Company |
| piperazinedione) | | |
| Docetaxel | Taxotere | Aventis |
| ((2R,3S)—N-carboxy-3-phenylisoserine, N-tert- | | Pharmaceuticals, Inc., |
| butyl ester, 13-ester with 5b-20-epoxy- | | Bridgewater, NJ |
| 12a,4,7b,10b,13a-hexahydroxytax-11-en-9-one 4- | | |
| acetate 2-benzoate, trihydrate) | | |
| Doxorubicin HCl | Adriamycin, | Pharmacia & Upjohn |
| (8S,10S)-10-[(3-amino-2,3,6-trideoxy-a-L-lyxo- | Rubex | Company |
| hexopyranosyl)oxy]-8-glycolyl-7,8,9,10- | | |
| tetrahydro-6,8,11-trihydroxy-1-methoxy-5,12- | | |
| naphthacenedione hydrochloride) | | |
| doxorubicin | Adriamycin PFS | Pharmacia & Upjohn |
| | Intravenous | Company |
| | injection | |
| doxorubicin liposomal | Doxil | Sequus |
| | | Pharmaceuticals, Inc., |
| | | Menlo park, CA |
| dromostanolone propionate | Dromostanolone | Eli Lilly & Company, |
| (17b-Hydroxy-2a-methyl-5a-androstan-3-one | | Indianapolis, IN |
| propionate) | | |
| dromostanolone propionate | Masterone | Syntex, Corp., Palo |
| | injection | Alto, CA |
| Elliott's B Solution | Elliott's | B Orphan Medical, Inc |
| | Solution | |
| Epirubicin | Ellence | Pharmacia & Upjohn |
| ((8S-cis)-10-[(3-amino-2,3,6-trideoxy-a-L- | | Company |
| arabino-hexopyranosyl)oxy]-7,8,9,10-tetrahydro- | | |
| 6,8,11-trihydroxy-8-(hydroxyacetyl)-1-methoxy- | | |
| 5,12-naphthacenedione hydrochloride) | | |
| Epoetin alfa | Epogen | Amgen, Inc |
| (recombinant peptide) | | |
| Estramustine | Emcyt | Pharmacia & Upjohn |
| (estra-1,3,5(10)-triene-3,17-diol(17(beta))-, 3- | | Company |
| [bis(2-chloroethyl)carbamate] 17-(dihydrogen | | |
| phosphate), disodium salt, monohydrate, or | | |
| estradiol 3-[bis(2-chloroethyl)carbamate] 17- | | |
| (dihydrogen phosphate), disodium salt, | | |
| monohydrate) | | |
| Etoposide phosphate | Etopophos | Bristol-Myers Squibb |
| (4′-Demethylepipodophyllotoxin 9-[4,6-O—(R)- | | |
| ethylidene-(beta)-D-glucopyranoside], 4′- | | |
| (dihydrogen phosphate)) | | |
| etoposide, VP-16 | Vepesid | Bristol-Myers Squibb |
| (4′-demethylepipodophyllotoxin 9-[4,6-0-(R)- | | |
| ethylidene-(beta)-D-glucopyranoside]) | | |
| Exemestane | Aromasin | Pharmacia & Upjohn |
| (6-methylenandrosta-1,4-diene-3,17-dione) | | Company |
| Filgrastim | Neupogen | Amgen, Inc |
| (r-metHuG-CSF) | | |
| floxuridine (intraarterial) | FUDR | Roche |
| (2′-deoxy-5-fluorouridine) | | |
| Fludarabine | Fludara | Berlex Laboratories, |
| (fluorinated nucleotide analog of the antiviral | | Inc., Cedar Knolls, |
| agent vidarabine, 9-b-D-arabinofuranosyladenine | | NJ |
| (ara-A)) | | |
| Fluorouracil, 5-FU | Adrucil | ICN Pharmaceuticals, |
| (5-fluoro-2,4(1H,3H)-pyrimidinedione) | | Inc., Humacao, |
| | | Puerto Rico |
| Fulvestrant | Faslodex | IPR Pharmaceuticals, |
| (7-alpha-[9-(4,4,5,5,5-penta fluoropentylsulphinyl) | | Guayama, Puerto |
| nonyl]estra-1,3,5-(10)-triene-3,17-beta-diol) | | Rico |
| Gemcitabine | Gemzar | Eli Lilly |
| (2′-deoxy-2′,2′-difluorocytidine | | |
| monohydrochloride (b-isomer)) | | |
| Gemtuzumab Ozogamicin | Mylotarg | Wyeth Ayerst |
| (anti-CD33 hP67.6) | | |
| Goserelin acetate | Zoladex Implant | AstraZeneca |
| (acetate salt of [D-Ser(But)⁶,Azgly¹⁰]LHRH; pyro- | | Pharmaceuticals |
| Glu-His-Trp-Ser-Tyr-D-Ser(But)-Leu-Arg-Pro- | | |
| Azgly-NH2 acetate [C₅₉H₈₄N₁₈O₁₄•(C₂H₄O₂)ₓ | | |
| Hydroxyurea | Hydrea | Bristol-Myers Squibb |
| Ibritumomab Tiuxetan | Zevalin | Biogen IDEC, Inc., |
| (immunoconjugate resulting from a thiourea | | Cambridge MA |
| covalent bond between the monoclonal antibody | | |
| Ibritumomab and the linker-chelator tiuxetan [N- | | |
| [2-bis(carboxymethyl)amino]-3-(p- | | |
| isothiocyanatophenyl)-propyl]-[N-[2- | | |
| bis(carboxymethyl)amino]-2-(methyl)- | | |
| ethyl]glycine) | | |
| Idarubicin | Idamycin | Pharmacia & Upjohn |
| (5,12-Naphthacenedione, 9-acetyl-7-[(3-amino- | | Company |
| 2,3,6-trideoxy-(alpha)-L-lyxo- | | |
| hexopyranosyl)oxy]-7,8,9,10-tetrahydro-6,9,11- | | |
| trihydroxyhydrochloride, (7S-cis)) | | |
| Ifosfamide | IFEX | Bristol-Myers Squibb |
| (3-(2-chloroethyl)-2-[(2- | | |
| chloroethyl)amino]tetrahydro-2H-1,3,2- | | |
| oxazaphosphorine 2-oxide) | | |
| Imatinib Mesilate | Gleevec | Novartis AG, Basel, |
| (4-[(4-Methyl-1-piperazinyl)methyl]-N-[4-methyl- | | Switzerland |
| 3-[[4-(3-pyridinyl)-2-pyrimidinyl]amino]- | | |
| phenyl]benzamide methanesulfonate) | | |
| Interferon alfa-2a | Roferon-A | Hoffmann-La Roche, |
| (recombinant peptide) | | Inc., Nutley, NJ |
| Interferon alfa-2b | Intron A | Schering AG, Berlin, |
| (recombinant peptide) | (Lyophilized | Germany |
| | Betaseron) | |
| Irinotecan HCl | Camptosar | Pharmacia & Upjohn |
| ((4S)-4,11-diethyl-4-hydroxy-9-[(4-piperidinopiperidino) | | Company |
| carbonyloxy]-1H-pyrano[3′,4′: | | |
| 6,7] indolizino[1,2-b] quinoline-3,14(4H,12H) | | |
| dione hydrochloride trihydrate) | | |
| Letrozole | Femara | Novartis |
| (4,4′-(1H-1,2,4-Triazol-1-ylmethylene) | | |
| dibenzonitrile) | | |
| Leucovorin | Wellcovorin, | Immunex, Corp., |
| (L-Glutamic acid, N[4[[(2amino-5-formyl- | Leucovorin | Seattle, WA |
| 1,4,5,6,7,8 hexahydro4oxo6- | | |
| pteridinyl)methyl]amino]benzoyl], calcium salt | | |
| (1:1)) | | |
| Levamisole HCl | Ergamisol | Janssen Research |
| ((—)-(S)-2,3,5,6-tetrahydro-6-phenylimidazo [2,1- | | Foundation, |
| b] thiazole monohydrochloride C₁₁H₁₂N₂S•HCl) | | Titusville, NJ |
| Lomustine | CeeNU | Bristol-Myers Squibb |
| (1-(2-chloro-ethyl)-3-cyclohexyl-1-nitrosourea) | | |
| Meclorethamine, nitrogen mustard | Mustargen | Merck |
| (2-chloro-N-(2-chloroethyl)-N-methylethanamine | | |
| hydrochloride) | | |
| Megestrol acetate | Megace | Bristol-Myers Squibb |
| 17α(acetyloxy)-6-methylpregna-4,6-diene- | | |
| 3,20-dione | | |
| Melphalan, L-PAM | Alkeran | GlaxoSmithKline |
| (4-[bis(2-chloroethyl)amino]-L-phenylalanine) | | |
| Mercaptopurine, 6-MP | Purinethol | GlaxoSmithKline |
| (1,7-dihydro-6H-purine-6-thione monohydrate) | | |
| Mesna | Mesnex | Asta Medica |
| (sodium 2-mercaptoethane sulfonate) | | |
| Methotrexate | Methotrexate | Lederle Laboratories |
| (N-[4-[[(2,4-diamino-6- | | |
| pteridinyl)methyl]methylamino]benzoyl]-L- | | |
| glutamic acid) | | |
| Methoxsalen | Uvadex | Therakos, Inc., Way |
| (9-methoxy-7H-furo[3,2-g][1]-benzopyran-7-one) | | Exton, Pa |
| Mitomycin C | Mutamycin | Bristol-Myers Squibb |
| mitomycin C | Mitozytrex | SuperGen, Inc., |
| | | Dublin, CA |
| Mitotane | Lysodren | Bristol-Myers Squibb |
| (1,1-dichloro-2-(o-chlorophenyl)-2-(p- | | |
| chlorophenyl) ethane) | | |
| Mitoxantrone | Novantrone | Immunex |
| (1,4-dihydroxy-5,8-bis[[2-[(2- | | Corporation |
| hydroxyethyl)amino]ethyl]amino]-9,10- | | |
| anthracenedione dihydrochloride) | | |
| Nandrolone phenpropionate | Durabolin-50 | Organon, Inc., West |
| | | Orange, NJ |
| Nofetumomab | Verluma | Boehringer Ingelheim |
| | | Pharma KG, |
| | | Germany |
| Oprelvekin | Neumega | Genetics Institute, |
| (IL-11) | | Inc., Alexandria, VA |
| Oxaliplatin | Eloxatin | Sanofi Synthelabo, |
| (cis-[(1R,2R)-1,2-cyclohexanediamine-N,N′] | | Inc., NY, NY |
| [oxalato(2-)-O,O′] platinum) | | |
| Paclitaxel | TAXOL | Bristol-Myers Squibb |
| (5β,20-Epoxy-1,2a,4,7β,10β,13a- | | |
| hexahydroxytax-11-en-9-one 4,10-diacetate 2- | | |
| benzoate 13-ester with (2R,3S)—N-benzoyl-3- | | |
| phenylisoserine) | | |
| Pamidronate | Aredia | Novartis |
| (phosphonic acid (3-amino-1-hydroxypropylidene) | | |
| bis-, disodium salt, pentahydrate, (APD)) | | |
| Pegademase | Adagen | Enzon |
| ((monomethoxypolyethylene glycol succinimidyl) | (Pegademase | Pharmaceuticals, Inc., |
| 11-17-adenosine deaminase) | Bovine) | Bridgewater, NJ |
| Pegaspargase | Oncaspar | Enzon |
| (monomethoxypolyethylene glycol succinimidyl | | |
| L-asparaginase) | | |
| Pegfilgrastim | Neulasta | Amgen, Inc |
| (covalent conjugate of recombinant methionyl | | |
| human G-CSF (Filgrastim) and | | |
| monomethoxypolyethylene glycol) | | |
| Pentostatin | Nipent | Parke-Davis |
| | | Pharmaceutical Co., |
| | | Rockville, MD |
| Pipobroman | Vercyte | Abbott Laboratories, |
| | | Abbott Park, IL |
| Plicamycin, Mithramycin | Mithracin | Pfizer, Inc., NY, NY |
| (antibiotic produced by Streptomyces plicatus) | | |
| Porfimer sodium | Photofrin | QLT |
| | | Phototherapeutics, |
| | | Inc., Vancouver, |
| | | Canada |
| Procarbazine | Matulane | Sigma Tau |
| (N-isopropyl-μ-(2-methylhydrazino)-p-toluamide | | Pharmaceuticals, Inc., |
| monohydrochloride) | | Gaithersburg, MD |
| Quinacrine | Atabrine | Abbott Labs |
| (6-chloro-9-(1-methyl-4-diethyl-amine) | | |
| butylamino-2-methoxyacridine) | | |
| Rasburicase | Elitek | Sanofi-Synthelabo, |
| (recombinant peptide) | | Inc., |
| Rituximab | Rituxan | Genentech, Inc., |
| (recombinant anti-CD20 antibody) | | South San Francisco, |
| | | CA |
| Sargramostim | Prokine | Immunex Corp |
| (recombinant peptide) | | |
| Streptozocin | Zanosar | Pharmacia & Upjohn |
| (streptozocin 2-deoxy-2- | | Company |
| [[(methylnitrosoamino)carbonyl]amino]-a(and b)- | | |
| D-glucopyranose and 220 mg citric acid | | |
| anhydrous) | | |
| Talc | Sclerosol | Bryan, Corp., |
| (Mg₃Si₄O₁₀(OH)₂) | | Woburn, MA |
| Tamoxifen | Nolvadex | AstraZeneca |
| ((Z)2-[4-(1,2-diphenyl-1-butenyl) phenoxy]-N,N- | | Pharmaceuticals |
| dimethylethanamine 2-hydroxy-1,2,3- | | |
| propanetricarboxylate (1:1)) | | |
| Temozolomide | Temodar | Schering |
| (3,4-dihydro-3-methyl-4-oxoimidazo[5,1-d]-as- | | |
| tetrazine-8-carboxamide) | | |
| teniposide, VM-26 | Vumon | Bristol-Myers Squibb |
| (4′-demethylepipodophyllotoxin 9-[4,6-0-(R)-2- | | |
| thenylidene-(beta)-D-glucopyranoside]) | | |
| Testolactone | Teslac | Bristol-Myers Squibb |
| (13-hydroxy-3-oxo-13,17-secoandrosta-1,4-dien- | | |
| 17-oic acid [dgr]-lactone) | | |
| Thioguanine, 6-TG | Thioguanine | GlaxoSmithKline |
| (2-amino-1,7-dihydro-6H-purine-6-thione) | | |
| Thiotepa | Thioplex | Immunex |
| (Aziridine, 1,1′,1″-phosphinothioylidynetris-, or | | Corporation |
| Tris (1-aziridinyl) phosphine sulfide) | | |
| Topotecan HCl | Hycamtin | GlaxoSmithKline |
| ((S)-10-[(dimethylamino) methyl]-4-ethyl-4,9- | | |
| dihydroxy-1H-pyrano[3′,4′:6,7] indolizino [1,2-b] | | |
| quinoline-3,14-(4H,12H)-dione | | |
| monohydrochloride) | | |
| Toremifene | Fareston | Roberts |
| (2-(p-[(Z)-4-chloro-1,2-diphenyl-1-butenyl]- | | Pharmaceutical |
| phenoxy)-N,N-dimethylethylamine citrate (1:1)) | | Corp., Eatontown, NJ |
| Tositumomab, I 131 Tositumomab | Bexxar | Corixa Corp., Seattle, |
| (recombinant murine immunotherapeutic | | WA |
| monoclonal IgG₂ₐ lambda anti-CD20 antibody (I | | |
| 131 is a radioimmunotherapeutic antibody)) | | |
| Trastuzumab | Herceptin | Genentech, Inc |
| (recombinant monoclonal IgG₁ kappa anti-HER2 | | |
| antibody) | | |
| Tretinoin, ATRA | Vesanoid | Roche |
| (all-trans retinoic acid) | | |
| Uracil Mustard | Uracil Mustard | Roberts Labs |
| | Capsules | |
| Valrubicin, N-trifluoroacetyladriamycin-14- | Valstar | Anthra --> Medeva |
| valerate | | |
| ((2S-cis)-2-[1,2,3,4,6,11-hexahydro-2,5,12- | | |
| trihydroxy-7 methoxy-6,11-dioxo-[[4 2,3,6- | | |
| trideoxy-3-[(trifluoroacetyl)-amino-α-L-lyxo- | | |
| hexopyranosyl]oxyl]-2-naphthacenyl]-2-oxoethyl | | |
| pentanoate) | | |
| Vinblastine, Leurocristine | Velban | Eli Lilly |
| (C₄₆H₅₆N₄O₁₀•H₂SO₄) | | |
| Vincristine | Oncovin | Eli Lilly |
| (C₄₆H₅₆N₄O₁₀•H₂SO₄) | | |
| Vinorelbine | Navelbine | GlaxoSmithKline |
| (3′,4′-didehydro-4′-deoxy-C′- | | |
| norvincaleukoblastine [R-(R*,R*)-2,3- | | |
| dihydroxybutanedioate (1:2)(salt)]) | | |
| Zoledronate, Zoledronic acid | Zometa | Novartis |
| ((1-Hydroxy-2-imidazol-1-yl-phosphonoethyl) | | |
| phosphonic acid monohydrate) | | |

Antimicrobial therapeutic agents may also be used as therapeutic agents in the present invention. Any agent that can kill, inhibit, or otherwise attenuate the function of microbial organisms may be used, as well as any agent contemplated to have such activities. Antimicrobial agents include, but are not limited to, natural and synthetic antibiotics, antibodies, inhibitory proteins (e.g., defensins), antisense nucleic acids, membrane disruptive agents and the like, used alone or in combination. Indeed, any type of antibiotic may be used including, but not limited to, antibacterial agents, antiviral agents, antifungal agents, and the like.

In still further embodiments, the present invention provides compounds of the present invention (and any other chemotherapeutic agents) associated with targeting agents that are able to specifically target particular cell types (e.g., tumor cells). Generally, the therapeutic compound that is associated with a targeting agent, targets neoplastic cells through interaction of the targeting agent with a cell surface moiety that is taken into the cell through receptor mediated endocytosis.

Any moiety known to be located on the surface of target cells (e.g., tumor cells) finds use with the present invention. For example, an antibody directed against such a moiety targets the compositions of the present invention to cell surfaces containing the moiety. Alternatively, the targeting moiety may be a ligand directed to a receptor present on the cell surface or vice versa. Similarly, vitamins also may be used to target the therapeutics of the present invention to a particular cell.

As used herein, the term “targeting molecules” refers to chemical moieties, and portions thereof useful for targeting therapeutic compounds to cells, tissues, and organs of interest. Various types of targeting molecules are contemplated for use with the present invention including, but not limited to, signal peptides, antibodies, nucleic acids, toxins and the like. Targeting moieties may additionally promote the binding of the associated chemical compounds (e.g., small molecules) or the entry of the compounds into the targeted cells, tissues, and organs. Preferably, targeting moieties are selected according to their specificity, affinity, and efficacy in selectively delivering attached compounds to targeted sites within a subject, tissue, or a cell, including specific subcellular locations and organelles.

Various efficiency issues affect the administration of all drugs—and of highly cytotoxic drugs (e.g., anticancer drugs) in particular. One issue of particular importance is ensuring that the administered agents affect only targeted cells (e.g., cancer cells), tissues, or organs. The nonspecific or unintended delivery of highly cytotoxic agents to nontargeted cells can cause serious toxicity issues.

Numerous attempts have been made to devise drug-targeting schemes to address the problems associated with nonspecific drug delivery. (See e.g., K. N. Syrigos and A. A. Epenetos Anticancer Res., 19:606-614 (1999); Y. J. Park et al., J. Controlled Release, 78:67-79 (2002); R. V. J. Chari, Adv. Drug Deliv. Rev., 31:89-104 (1998); and D. Putnam and J. Kopecek, Adv. Polymer Sci., 122:55-123 (1995)). Conjugating targeting moieties such as antibodies and ligand peptides (e.g., RDG for endothelium cells) to drug molecules has been used to alleviate some collateral toxicity issues associated with particular drugs.

The compounds and anticancer agents may be administered in any sterile, biocompatible pharmaceutical carrier, including, but not limited to, saline, buffered saline, dextrose, and water. In some embodiments, the pharmaceutical compositions of the present invention may contain one agent (e.g., an antibody). In other embodiments, the pharmaceutical compositions contain a mixture of at least two agents (e.g., an antibody and one or more conventional anticancer agents). In still further embodiments, the pharmaceutical compositions of the present invention contain at least two agents that are administered to a patient under one or more of the following conditions: at different periodicities, at different durations, at different concentrations, by different administration routes, etc. In some embodiments, the growth hormone receptor antagonist is administered prior to the second anticancer agent, e.g., 0.5, 1, 2 3, 4, 5, 10, 12, or 18 hours, 1, 2, 3, 4, 5, or 6 days, 1, 2, 3, or 4 weeks prior to the administration of the anticancer agent. In some embodiments, the growth hormone receptor antagonist is administered after the second anticancer agent, e.g., 0.5, 1, 2 3, 4, 5, 10, 12, or 18 hours, 1, 2, 3, 4, 5, or 6 days, 1, 2, 3, or 4 weeks after the administration of the anticancer agent. In some embodiments, the growth hormone receptor antagonist and the second anticancer agent are administered concurrently but on different schedules, e.g., the growth hormone receptor antagonist compound is administered daily while the second anticancer agent is administered once a week, once every two weeks, once every three weeks, or once every four weeks. In other embodiments, the growth hormone receptor antagonist is administered once a week while the second anticancer agent is administered daily, once a week, once every two weeks, once every three weeks, or once every four weeks.

Depending on the condition being treated, preferred embodiments of the present pharmaceutical compositions are formulated and administered systemically or locally. Techniques for formulation and administration can be found in the latest edition of “Remington's Pharmaceutical Sciences” (Mack Publishing Co, Easton Pa.). Suitable routes may, for example, include oral or transmucosal administration as well as parenteral delivery (e.g., intramuscular, subcutaneous, intramedullary, intrathecal, intraventricular, intravenous, intraperitoneal, or intranasal administration).

The present invention contemplates administering therapeutic compounds and, in some embodiments, one or more conventional anticancer agents, in accordance with acceptable pharmaceutical delivery methods and preparation techniques. For example, therapeutic compounds and suitable anticancer agents can be administered to a subject intravenously in a pharmaceutically acceptable carrier such as physiological saline. Standard methods for intracellular delivery of pharmaceutical agents are contemplated (e.g., delivery via liposome). Such methods are well known to those of ordinary skill in the art.

In some embodiments, the formulations of the present invention are useful for parenteral administration (e.g., intravenous, subcutaneous, intramuscular, intramedullary, and intraperitoneal). Therapeutic co-administration of some contemplated anticancer agents (e.g., therapeutic polypeptides) can also be accomplished using gene therapy reagents and techniques.

In some embodiments of the present invention, therapeutic compounds are administered to a subject alone, or in combination with one or more conventional anticancer agents (e.g., nucleotide sequences, drugs, hormones, etc.) or in pharmaceutical compositions where the components are optionally mixed with excipient(s) or other pharmaceutically acceptable carriers. In preferred embodiments of the present invention, pharmaceutically acceptable carriers are biologically inert. In preferred embodiments, the pharmaceutical compositions of the present invention are formulated using pharmaceutically acceptable carriers well known in the art in dosages suitable for oral administration. Such carriers enable the pharmaceutical compositions to be formulated as tablets, pills, capsules, dragees, liquids, gels, syrups, slurries, solutions, suspensions and the like, for respective oral or nasal ingestion by a subject.

Pharmaceutical preparations for oral use can be obtained by combining the active compounds with solid excipients, optionally grinding the resulting mixture, and processing the mixture into granules, after adding suitable auxiliaries, if desired, to obtain tablets or dragee cores. Suitable excipients are carbohydrate or protein fillers such as sugars, including lactose, sucrose, mannitol, or sorbitol; starch from corn, wheat, rice, potato, etc.; cellulose such as methyl cellulose, hydroxypropylmethyl-cellulose, or sodium carboxymethylcellulose; gums including arabic and tragacanth; and proteins such as gelatin and collagen. If desired, disintegrating or solubilizing agents may be added, such as the cross-linked polyvinyl pyrrolidone, agar, alginic acid or a salt thereof such as sodium alginate.

In preferred embodiments, dosing and administration regimes are tailored by the clinician, or others skilled in the pharmacological arts, based upon well known pharmacological and therapeutic considerations including, but not limited to, the desired level of therapeutic effect, and the practical level of therapeutic effect obtainable. Generally, it is advisable to follow well-known pharmacological principles for administrating chemotherapeutic agents (e.g., it is generally advisable to not change dosages by more than 50% at time and no more than every 3-4 agent half-lives). For compositions that have relatively little or no dose-related toxicity considerations, and where maximum efficacy (e.g., destruction of cancer cells) is desired, doses in excess of the average required dose are not uncommon. This approach to dosing is commonly referred to as the “maximal dose” strategy. In certain embodiments, the growth hormone receptor antagonist is administered to a subject at a dose of 1-40 mg per day (e.g. for 4-6 weeks). In certain embodiments, subject is administered a loading dose of between 15-70 mg of the growth hormone receptor antagonist. In certain embodiments, the subject is administered a loading dose of about 35-45 mg of the growth hormone receptor antagonist (e.g. subcutaneously), and then daily doses of about 10 mg (e.g. subcutaneously) for about 4-6 weeks.

Additional dosing considerations relate to calculating proper target levels for the agent being administered, the agent's accumulation and potential toxicity, stimulation of resistance, lack of efficacy, and describing the range of the agent's therapeutic index.

In certain embodiments, the present invention contemplates using routine methods of titrating the agent's administration. One common strategy for the administration is to set a reasonable target level for the agent in the subject. In some preferred embodiments, agent levels are measured in the subject's plasma. Proper dose levels and frequencies are then designed to achieve the desired steady-state target level for the agent. Actual, or average, levels of the agent in the subject are monitored (e.g., hourly, daily, weekly, etc.) such that the dosing levels or frequencies can be adjusted to maintain target levels. Of course, the pharmacokinetics and pharmacodynamics (e.g., bioavailability, clearance or bioaccumulation, biodistribution, drug interactions, etc.) of the particular agent or agents being administered can potentially impact what are considered reasonable target levels and thus impact dosing levels or frequencies.

Target-level dosing methods typically rely upon establishing a reasonable therapeutic objective defined in terms of a desirable range (or therapeutic range) for the agent in the subject. In general, the lower limit of the therapeutic range is roughly equal to the concentration of the agent that provides about 50% of the maximum possible therapeutic effect. The upper limit of the therapeutic range is usually established by the agent's toxicity and not by its efficacy. The present invention contemplates that the upper limit of the therapeutic range for a particular agent will be the concentration at which less than 5 or 10% of subjects exhibit toxic side effects. In some embodiments, the upper limit of the therapeutic range is about two times, or less, than the lower limit. Those skilled in the art will understand that these dosing consideration are highly variable and to some extent individualistic (e.g., based on genetic predispositions, immunological considerations, tolerances, resistances, and the like). Thus, in some embodiments, effective target dosing levels for an agent in a particular subject may be 1, . . . 5, . . . 10, . . . 15, . . . 20, . . . 50, . . . 75, . . . 100, . . . 200, . . . X %, greater than optimal in another subject. Conversely, some subjects may suffer significant side effects and toxicity related health issues at dosing levels or frequencies far less (1, . . . 5, . . . 10, . . . 15, . . . 20, . . . 50, . . . 75, . . . 100, . . . 200, . . . X %) than those typically producing optimal therapeutic levels in some or a majority of subjects. In the absence of more specific information, target administration levels are often set in the middle of the therapeutic range.

In preferred embodiments, the clinician rationally designs an individualized dosing regimen based on known pharmacological principles and equations. In general, the clinician designs an individualized dosing regimen based on knowledge of various pharmacological and pharmacokinetic properties of the agent, including, but not limited to, F (fractional bioavailability of the dose), Cp (concentration in the plasma), CL (clearance/clearance rate), Vss (volume of drug distribution at steady state) Css (concentration at steady state), and t½ (drug half-life), as well as information about the agent's rate of absorption and distribution. Those skilled in the art are referred to any number of well known pharmacological texts (e.g., Goodman and Gilman's, Pharmaceutical Basis of Therapeutics, 10th ed., Hardman et al., eds., 2001) for further explanation of these variables and for complete equations illustrating the calculation of individualized dosing regimes. Those skilled in the art also will be able to anticipate potential fluctuations in these variables in individual subjects. For example, the standard deviation in the values observed for F, CL, and Vss is typically about 20%, 50%, and 30%, respectively. The practical effect of potentially widely varying parameters in individual subjects is that 95% of the time the Css achieved in a subject is between 35 and 270% that of the target level. For drugs with low therapeutic indices, this is an undesirably wide range. Those skilled in the art will appreciate, however, that once the agent's Cp (concentration in the plasma) is measured, it is possible to estimate the values of F, CL, and Vss directly. This allows the clinician to effectively fine tune a particular subject's dosing regimen.

In still other embodiments, the present invention contemplates that continuing therapeutic drug monitoring techniques be used to further adjust an individual's dosing methods and regimens. For example, in one embodiment, Css data is used is to further refine the estimates of CL/F and to subsequently adjust the individual's maintenance dosing to achieve desired agent target levels using known pharmacological principles and equations. Therapeutic drug monitoring can be conducted at practically any time during the dosing schedule. In preferred embodiments, monitoring is carried out at multiple time points during dosing and especially when administering intermittent doses. For example, drug monitoring can be conducted concomitantly, within fractions of a second, seconds, minutes, hours, days, weeks, months, etc., of administration of the agent regardless of the dosing methodology employed (e.g., intermittent dosing, loading doses, maintenance dosing, random dosing, or any other dosing method). However, those skilled in the art will appreciate that when sampling rapidly follows agent administration the changes in agent effects and dynamics may not be readily observable because changes in plasma concentration of the agent may be delayed (e.g., due to a slow rate of distribution or other pharmacodynamic factors). Accordingly, subject samples obtained shortly after agent administration may have limited or decreased value.

The primary goal of collecting biological samples from the subject during the predicted steady-state target level of administration is to modify the individual's dosing regimen based upon subsequently calculating revised estimates of the agent's CL/F ratio. However, those skilled in the art will appreciate that early postabsorptive drug concentrations do not typically reflect agent clearance. Early postabsorptive drug concentrations are dictated principally by the agent's rate of absorption, the central, rather than the steady state, volume of agent distribution, and the rate of distribution. Each of these pharmacokinetic characteristics have limited value when calculating therapeutic long-term maintenance dosing regimens.

Accordingly, in some embodiments, when the objective is therapeutic long-term maintenance dosing, biological samples are obtained from the subject, cells, or tissues of interest well after the previous dose has been administered, and even more preferably shortly before the next planned dose is administered.

In still other embodiments, where the therapeutic agent is nearly completely cleared by the subject in the interval between doses, then the present invention contemplates collecting biological samples from the subject at various time points following the previous administration, and most preferably shortly after the dose was administered.

### EXAMPLES

The following example is provided in order to demonstrate and further illustrate certain preferred embodiments and aspects of the present invention and is not to be construed as limiting the scope thereof.

### Example 1

### Growth Hormone and Growth Hormone Antagonist Treatment of Progenitor Mammary Cells

This example describes methods of assaying the impact of growth hormone and a growth hormone antagonist on cultured progenitor mammary cells. Previous studies have compared the gene expression profile of undifferentiated cells grown as mammospheres to that of differentiated cells cultured on collagen, utilizing AFFYMETRIX microarray analysis (Dontu et al., Genes & Development, 17(10); 1253-1270, 2003, herein incorporated by reference in its entirety for all purposes). In this study, primary mammospheres were dissociated to single cells and divided in two aliquots that were re-plated in suspension culture and on collagen, in differentiating conditions respectively, and total RNA was extracted from the second generation spheres and from cells grown under differentiating conditions and used in the hybridization reaction against the AFFYMETRIX chip. GRH (growth hormone receptor) was found to be up-regulated in undifferentiated mammary cells.

In this example, immuno-magnetic sorting and flow cytometry was utilized in combination with sphere formation to enrich for stem/progenitor cells. Cells were sorted according to the presence or absence of the marker and then each population assayed for its ability to form mammospheres. This procedure identified a number of markers that enrich in mammosphere initiating cells, one of which is growth hormone receptor (GHR) as shown in Table II. Some of these markers are enriched or expressed exclusively in cells grown as mammospheres and are also expressed in other stem cells, including hematopoietic, neuronal, and embryonic stem cells, as shown by the transcriptional profiling (Table II)

TABLE II
Mammosphere formation by immunosorted cells

Percentage positive
cells

| | | |
|---|---|---|
| Total | | #spheres/10,000 |
| HMEC | Immuno | cells |

| Antigen | population | sorted cells | Neg. | Pos. |
|---|---|---|---|---|
| CD44 | 40.4 | 95 | 5 | 40 |
| LIF-R | 14.8 | 77 | 5 | 164 |
| CXCR4 | 4.8 | 50 | 8 | 225 |
| GHR | 5.5 | 75 | 5 | 130 |
| gp 130 | 3.8 | 71 | 8 | 100 |

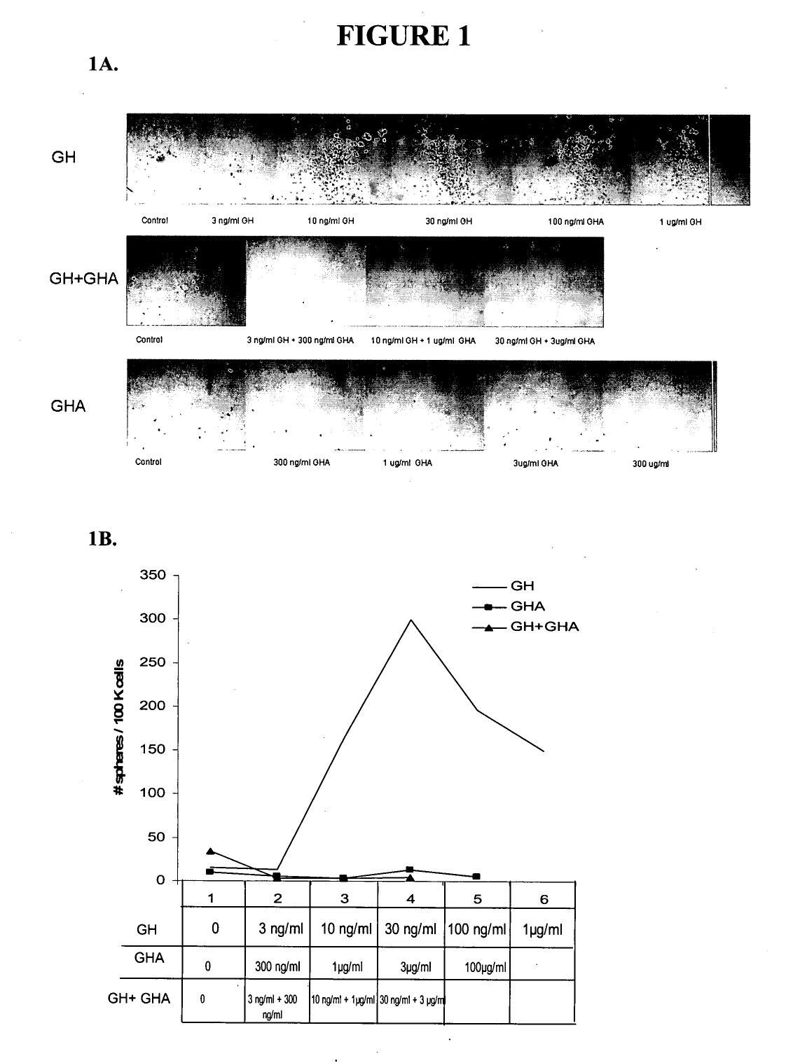

The role of GH signalling in mammary progenitor cells proliferation and differentiation was investigated by treating mammosphere cultures with either recombinant human GH or a synthetic peptide (Pegvisomant) that functions as an antagonist and assessed the effect on number and size of mammospheres. As shown in FIGS. 1A and 1B, GH treatment dramatically increases the survival and proliferation of mammary epithelial cells in suspension, as reflected by the increase in mammosphere size and numbers. The effect is specific, since it is inhibited by the antagonist treatment. The GHA treatment does not appear to affect cultures untreated with GH, suggesting that an autocrine mechanism is probably not involved.

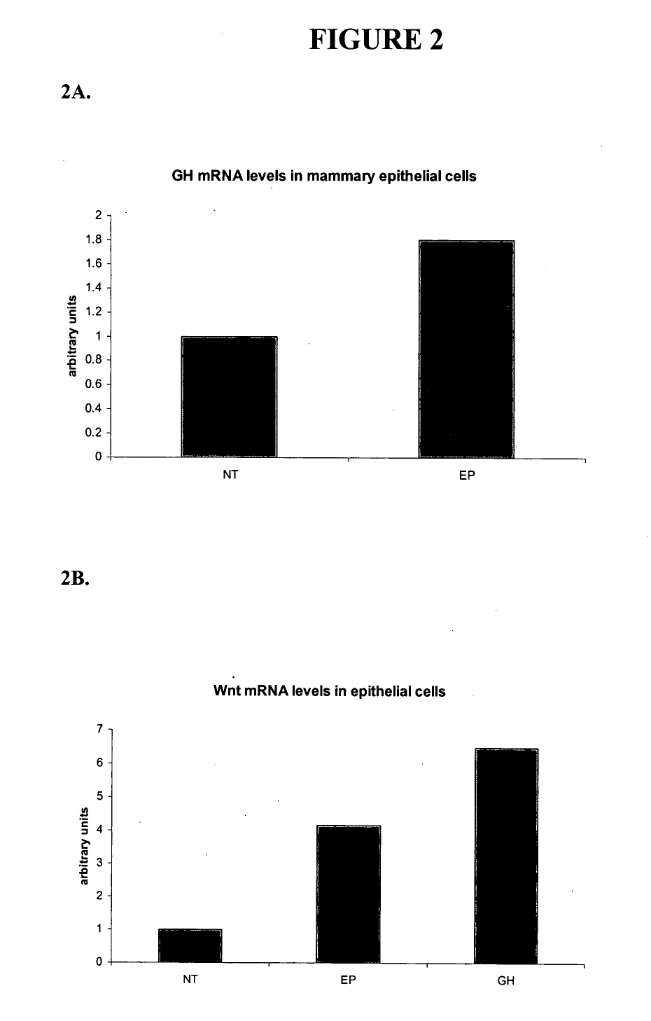

It was also found that growth hormone mRNA levels in normal mammary epithelial cells grown as mammospheres is considerably increased by treatment with estradiol and progesterone (FIG. 2A). Wnt1 mRNA level is also increased by estradiol progesterone treatment, but also by treatment with human recombinant growth hormone (FIG. 2B).

### Example 2

### GH is Produced by Mammary Epithelial Cells is Response to Estrogen and Progesterone Stimulation

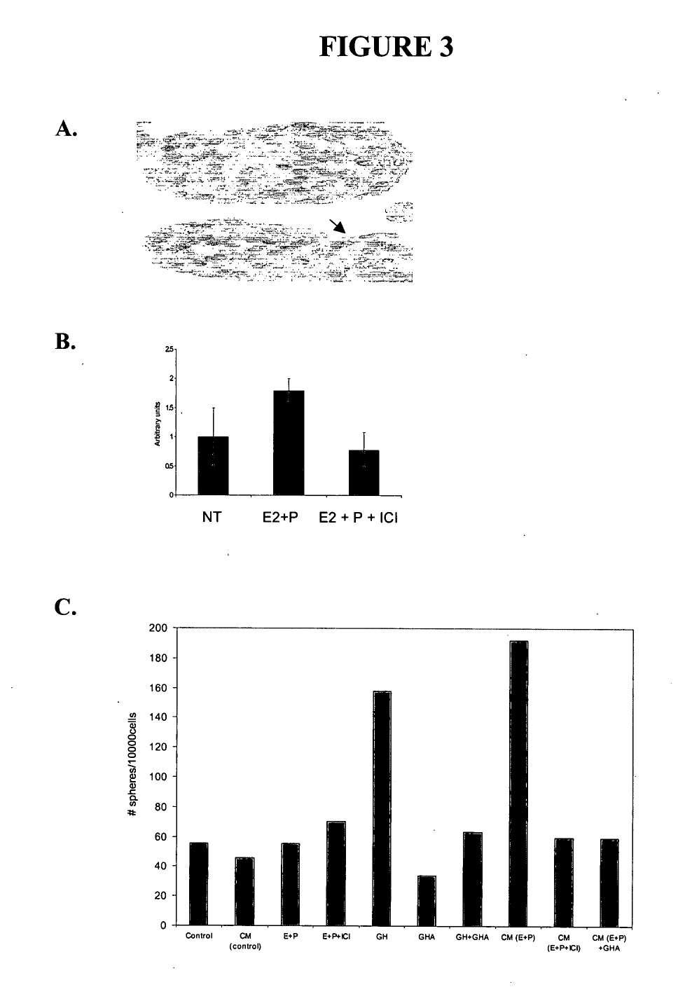

It has been determined that ER positive cells are present in the mammospheres (FIG. 3 A). The progesterone receptor (PR) is expressed in ER+ cells after estrogen stimulation and is activated by systemic progesterone. The increase in progesterone is slightly delayed compared to the increase in estrogen level, both in the first half of the menstrual cycle and during pregnancy. Cultures were simultaneously treated with beta-estradiol, progesterone and a derivate of progesterone with longer life in culture. It was determined that estradiol and progesterone stimulation (10-9 M beta-estradiol and same concentration progesterone) increased GH mRNA levels in human mammary epithelial cells (HMECs) grown as mammospheres. This effect was blocked by ICI 182,780 (Faslodex; AstraZenaca) (FIG. 3 B). These results were confirmed by testing GH protein levels in the concentrated culture medium by ELISA (data not shown). To investigate if the GH secreted by mammary epithelial cells has an effect on self-renewal, mammosphere cultures were treated with conditioned medium from cells treated with estradiol and progesterone for 24 hours. The results (shown in FIG. 3C) show that while, at the concentrations used (10-9 M), estradiol (E2) and progesterone (P) did not increase sphere formation in secondary passage, the conditioned medium from spheres treated in this manner, increased sphere formation. This effect was comparable to that achieved by exogenous stimulation with recombinant GH at a concentration of 10 ng/ml and it was abolished by GHA treatment. The effect was mediated by estradiol and progesterone, because conditioned medium from cultures treated with steroids and ICI 182,780 did not have an effect on sphere formation.

### Example 3

### Effect of GHA Treatment on Breast Cancer Cells

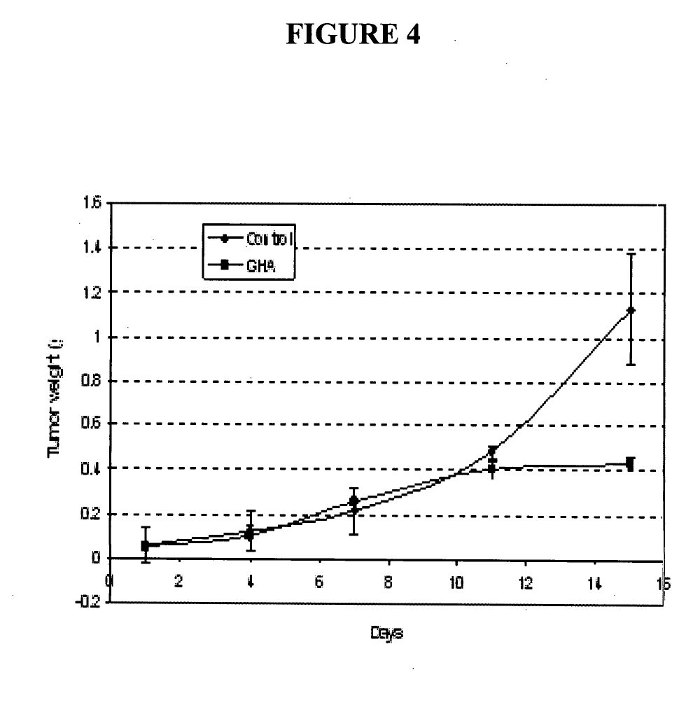

This example describes the effect of a growth hormone antagonist (GHA) on breast cancer cells in vivo. NOD/scid mice were implanted with human breast carcinoma cells and treated with Pegvisomant. The breast cancer cells employed were not grown in vitro. Treatment was started when tumors were 0.3/0.3 cm in diameter. Six animals per group of treatment were used. Pegvisomant was administered 20 mg/kg, 1/day, sc, for 2 weeks. As shown in FIG. 4, the results of this example show a considerable difference in tumor growth in the animals treated with the GHA compared to controls.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the present invention.

We claim:

1. A method of reducing or eliminating tumorigenic cells in a subject, comprising: administering a growth hormone receptor antagonist to said subject under conditions such that at least a portion of said tumorigenic cells are killed, inhibited from proliferating, or from causing metastasis.

2. The method of claim 1, wherein said growth hormone receptor antagonist comprises Pegvisomant.

3. The method of claim 1, wherein said tumorigenic cells are mammary progenitor cells.

4. The method of claim 1, wherein said growth hormone receptor antagonist comprises an antibody or antibody fragment.

5. The method of claim 1, wherein said growth hormone receptor antagonist comprises a modified form of the human growth hormone protein.

6. The method of claim 1, wherein said tumorigenic cells are mammary cells characterized by an increased level of expression of growth hormone receptor compared to non-tumorigenic mammary cells from said subject.

7. The method of claim 1, wherein said subject does not have acromegaly.

8. The method of claim 1, further comprising surgically removing a tumor from said subject prior to said administering.

9. A method for screening a compound, comprising: a) exposing a sample comprising a tumorigenic mammary cell to a candidate anti-neoplastic compound, wherein said candidate anti-neoplastic compound comprises a growth hormone receptor antagonist; and b) detecting a change in said cell in response to said compound.

10. The method of claim 9, wherein said sample comprises a non-adherent mammosphere.

11. The method of claim 9, wherein said growth hormone receptor antagonist comprises an antibody or antibody fragment.

12. The method of claim 9, wherein said growth hormone receptor antagonist comprises a modified form of the human growth hormone protein.

13. The method of claim 9, wherein said sample comprises human breast tissue.

14. The method of claim 9, wherein said detecting comprises detecting cell death of said tumorigenic breast cell.

15. The method of claim 14, further comprising identifying said candidate anti-neoplastic agent as capable of killing tumorigenic cells.

16. A method of obtaining an enriched population of progenitor cells, comprising a) providing an initial sample comprising progenitor and non-progenitor cells, and b) sorting said initial sample based on the growth hormone receptor (GHR) expression level in said cells such that an enriched population is generated, wherein said enriched population contains a higher percentage of progenitor cells than present in said initial sample.

17. The method of claim 16, wherein said sorting comprises the use of flow cytometry.

18. The method of claim 16, wherein said sorting comprises the use of immuno-magnetic sorting.

19. The method of claim 16, wherein said progenitor cells comprise tumorigenic cells and said non-progenitor cells comprise non-tumorigenic cells.

20. The method of claim 19, wherein said progenitor and non-progenitor cell, comprise mammary cells.
